(19) United States
(12) Patent Application Publication
(10) Pub. No.: US 2009/0169474 A1
(43) Pub. Date: Jul. 2, 2009

(54) METHOD FOR NONINVASNELY AND QUANTITATIVELY MONITORING THERAPEUTIC AND DIAGNOSTIC TRANSGENE EXPRESSION INDUCED BY EX VNO AND IN VNO GENE TARGETING IN ORGANS, TISSUES AND CELLS

(75) Inventors: Luyi Sen, Stevenson Ranch, CA (US); Sanjiv S. Gambhir, Portola Valley, CA (US)

Correspondence Address: Law Offices of Daniel L. Dawes, 5200 Warner Blvd, Ste. 106, Huntington Beach, CA, 92649, US

(73) Assignee: The Regentsf of the University of California, Oakland, CA (US)

(21) Appl. No.: 12/280,680

(22) PCT Filed: Mar. 20, 2007
(86) PCT No.: PCT/US07/07048

§ 371 Date: Oct. 20, 2008

Related U.S. Application Data
(60) Provisional application No. 60/743,620, filed on Mar. 21, 2006.

(51) Int. Cl. A61K 51/06; C12Q 1/68; A61K 49/04

(52) U.S. Cl. 424/1.73; 435/6; 424/9.4

(57) ABSTRACT

An composition in a method for noninvasively monitoring the expression of therapeutic transgene delivered ex vivo and in vivo for the treatment of diseases includes the step of quantitatively imaging a reporter gene expression which is coupled to a therapeutic gene on a plasmid vector to infer levels, location, or duration of the therapeutic gene expression in the targeted tissues or organs. The reporter gene is imaged using a radiopharmaceutical for scintigraphic imaging of the gene expression interactions with the reporter gene, namely positron emission tomography, gamma camera or single-photon emission computed tomography. The genes are delivered with a liposome encapsulated reporter-therapeutic linked transgene vector with balanced reporter/therapeutic transgene expression. A transgene composition includes the reporter gene linked to the therapeutic gene or genes incorporated in and delivered by a liposome encapsulated reporter-therapeutic linked transgene vector.

### RELATED APPLICATIONS

The present application is related to U.S. Provisional Patent Application Ser. No. 60/743,620 filed on Mar. 21, 2006, which is incorporated herein by reference and to which priority is claimed pursuant to 35 U.S.C. §119.

### BACKGROUND OF THE INVENTION

1. Field of the Invention

The invention relates to the field of noninvasively monitoring the expression of therapeutic and diagnostic transgene delivered ex vivo and in vivo for the treatment of any diseases in all organs, tissues and cells.

2. Description of the Prior Art

Gene therapy is ushering in a new era in the treatment of various inherited or acquired human diseases. However, its clinical application is limited by the lack of information of pharmacokinetics and pharmacodynamics. An essential technique is required to be able to determine the kinetics and distribution of the transgene expression in the targeted organ or tissue. To date, transgene expression can primarily be measured or imaged using fixed tissue obtained from postmortem or biopsy. A clinically applicable technique for noninvasively and quantitatively measuring the transgene expression level and distribution in the targeted organ or tissue is not available.

Recently, the transfer of one or two adenovirally mediated positron emission tomography (PET) reporter genes, most recently a reporter gene linked with a VEGF gene, into the rat heart has been accomplished by direct myocardial injection. Micro-PET images of focal distribution of a reporter gene in a very small region surrounding the injection site in rat and pig myocardium have been shown. However, the high titer of virus required for generating a detectable PET signal often induces significant inflammation in the myocardium and prevents long-term reporter gene expression. Although the principle of PET reporter gene imaging is promising, a safe and applicable approach to reporter-therapeutic gene delivery into whole heart and quantitative imaging of the therapeutic gene expression is still yet to be developed for clinical application. The lessons we learned from unsuccessful clinical trials in the past decade let us be aware of the urgent needs for a new and safer gene delivery strategy and better understanding of the pharmacodynamics and pharmacokinetics of gene therapy.

A clinically applicable noninvasive approach for assessing transgene expression is the key for both validating existing and new gene transfer strategies, and for developing and validating any clinical applicable new vectors and defining the success of transgene expression in target organs. However, such a clinically approach has not previously existed. In the experimental setting, assessment of gene expression is accomplished by in situ hybridization techniques or by using reporter genes that can be detected by various methods. For in vivo or ex vivo gene transfer studies, the assessment still has to be performed in post-mortem analysis or has required invasive procedures for tissue sampling.

Recent advances in the imaging of both green fluorescent protein and firefly luciferase for in vivo studies, using optical imaging devices, have made possible the use of optical techniques to analyze, in real time, reporter gene expression in rodents. This method is convenient and fast. Bioluminescence imaging is relatively more sensitive than fluorescence. However, the efficiency of light transmission is limited and depends on tissue type and tissue scattering. The net reduction of the bioluminescence signal is 10 fold for every centimeter of tissue depth for bioluminescence and this reduction is further doubled for fluorescence signal. The attenuation of visible light limits the use of this method in any animals larger than rodents. None of these methods can be used for humans.

One object of the illustrated embodiment of the invention is to establish a concept and clinically usable methodology for long-term noninvasively, quantitatively and repeatedly monitoring the magnitude, duration, and distribution of expression of any therapeutic transgene that is ex vivo or in vivo targeted in the various tissues and organs using any known transfection method, such as virus, liposome, electroporation, ultrasound, the like.

### BRIEF SUMMARY OF THE INVENTION

The illustrated embodiment of the invention includes two components: 1) a technology for long-term noninvasively, quantitatively and repeatedly monitoring the ex vivo targeted therapeutic transgene expression in various tissues and organs using reporter-therapeutic linked gene-probe with positron emission tomography, a gamma camera or single-photon emission computed tomography; and 2) a technology for long-term noninvasively, quantitatively and repeatedly monitoring the in vivo targeted therapeutic transgene expression in various cells, tissues and organs using reporter-therapeutic linked gene-probe with positron emission tomography, gamma camera or single-photon emission computed tomography. The apparatus and method of the invention can be used to monitor any organ, tissue, or cell gene therapy for both diagnostic and therapeutic gene transfer. One or more reporter genes can be used, which may be the same as or different than the therapeutic gene. The illustrated embodiment is used not only for liposome-mediate gene transfer, but for any other protocol of gene transfer as well. The gene may be on a plasmid, in the cell or in naked DNA.

The concept is to quantitatively image the reporter gene expression that is coupled to the therapeutic gene on the same plasmid vector to infer levels, location, and duration of therapeutic gene(s) expression in the targeted tissues or organs. This strategy requires proportional and constant co-expression of both the reporter gene and the therapeutic gene over a wide range of transgene expression levels. The principle of scintigraphic reporter gene-probe imaging is to use using radiopharmaceuticals for scintigraphic imaging of gene expression interactions with the reporter gene product. Positron emission tomography PET is the preferred scintigraphic imaging modality among other methods, due to its higher spatial resolution and higher sensitivity. The reporter gene encodes either for an enzyme that converts a radiolabelled substrate into a metabolite that in turn is exclusively trapped within cells expressing the reporter gene, or for a receptor that selectively binds radio-labelled ligands, or for a transmembrane carrier that results in selective uptake of radiolabelled nuclides.

Four major components included in the illustrated embodiment of the invention. The first component is based on the design of a reporter-therapeutic linked transgene vector with balanced reporter/therapeutic transgene(s) expression. The herpesviral thymidine kinase (HSV1-tk) is most popular enzyme reporter gene, but it also phosphorylates endogenous thymidine. It has been found that mutagenesis of the HSV1-tk active site (HSV1-sr39tk) results in an enzyme that could utilize acycloguanosine derivatives more effectively and endogenous thymidine less effectively than wild-type HSV1-tk. There are several strategies that have been validated to achieve linkage of expression of the therapeutic transgene and the imaging reporter gene. All of them were tested using virus vectors, because the transfection efficiency of naked plasmid/DNA was too low. We are the first to use two identical cytomegalovirus (CMV) promoters driving one reporter and one therapeutic gene in single plasmid. Our data shows a balanced reporter-therapeutic gene and therapeutic-therapeutic gene expression in the myocardium. Our results indicate that two identical promoters driven two genes in one vector did not interfere each other.

Recently we also developed a vector has one reporter gene and two therapeutic genes that can be used for monitoring combined gene therapy. On the other hand we also developed several different ways to optimize the vector design when the reporter and therapeutic gene expression are not balanced. Thus, this technique can be applicable for controlled gene therapy in various organ diseases using various therapeutic transgenes.

Although the autoimmunity is the major concern for the plasmid toxicity, to date there has been no convincing evidence of DNA vaccine-associated autoimmunity. Plasmid DNA thus can induce both cellular and humoral immune responses. The magnitude of these responses is generally modest when DNA is used alone. Primate studies and preliminary results of human trials suggest that more potent specific immune responses may be induced by combining DNA with adjuvants, by boosting with a recombinant viral vector or protein, or by both adjuvanting and boosting.

DNA is a complex macromolecule whose immunological properties vary with the base sequences. As shown with synthetic oligonucleotides, potent immune stimulation results from six base motifs called CpG motifs or immuno-stimulatory sequences (ISS). These sequences center on an unmethylated CpG dinucleotide and occur much more commonly in bacterial DNA than mammalian DNA. As such, CpG motifs may function as a danger signal to stimulate B lymphocyte cell activation and cytokine production. To reduce or eliminate the possible, but not yet confirmed, immunogenicity of the plasmid DNA, four strategies can be taken: 1) avoid the use of viral vectors; 2) use liposome encapsulation to reduce the immune response, interestingly, DNA complexed with liposome may also reduce the cytotoxicity of cationic liposome as we described in the above section; 3) avoid ectopic gene transfection by localized gene delivery in targeted organ or tissue; and 4) construct a CpG plasmid free vector. The ex vivo and in vivo gene transfer methods proposed in our study have been in compliance with the first three requirements. Most recently, we constructed a CpG free plasmid vector with HSV1-sr39tk and human IL-10 gene driven by two identical CpG free human elongation factor 1α (EF-1α) promoters, pCpGf-EF1HSV1sr39tk-EF1hIL-10.*

In contrast to the CMV vector, a most efficient but also the most immunogenic promoter, human EF-1α, is much less immunogenic, because it contains virtually no CpG. EF-1 promoter is manufactured by Invivogene, San Diego, Calif., displays a strong activity and yields persistent expression in vivo. We are currently validating this vector using our liposome-mediated ex vivo intracoronary gene transfer method in the rabbit isograft transplantation model. We have validated this vector using our ex vivo intracoronary gene delivery method in rabbit heart transplant model in 6 rabbit cardiac isografts. Our preliminary data have already shown that the transgene expression induced by this new vector is significantly higher than the conventional plasmid with two CMV promoters. A balanced reporter and therapeutic gene expression was observed. There is no cardiac adverse effect and immunogenicity found in these rabbits.

The second major component of the illustrated embodiment of the invention is that the reporter probe has no effect on the host cell metabolism and function, but has high specificity for the binding effect with the isotope for imaging.

Two main categories of substrates, uracil nucleoside derivatives labelled with radioactive iodine (e.g., I-labelled 2′fluoro-2′-deoxy-1-β-D-arabinofura-nosyl-5-iodo-uracilc(FIAU)), and acycloguanosine derivatives labelled with radioactive ¹⁸F-fluorine (e.g., 9-[(4-[¹⁸F]-fluoro-3-hydroxymethylbutyl)guanine (FHBG) and 8-[¹⁸F]fluropenciclorivr (FPCV)), have been investigated as reporter probes for imaging HSV1-tk reporter gene expression. We found FHBG to be a more effective probe for in vivo imaging of the wild-type HSV1-tk and HSV1-sr39tk PET reporter genes than FPCV.

The third major component of the illustrated embodiment of the invention is the clinically applicable ex vivo and in vivo reporter and therapeutic linked gene delivery systems in various organs and tissues of large animals and humans.

Most reporter gene imaging studies were either in vitro or in vivo by injecting the gene into tumors. To date, little information is available for noninvasive imaging of transgene expression in the heart. In all of the prior studies, the reporter gene was directly injected in the myocardium. These studies are just focused on qualitatively imaging the regional reporter gene expression in the injection site.

We have developed ex vivo and in vivo gene delivery strategies in the whole heart of large animals. We previously developed a rabbit heterotopic functional heart transplant/acute cardiac rejection model that is the only functional heart transplant animal model available to date and has been used for validated various gene transfer techniques and therapeutic genes. The ex vivo intracoronary delivered and liposome-mediated IL-10 gene therapy approach we previously developed has been the most well characterized nonviral gene therapy model, that could reproducibly induce localized immuno-suppression and prolongs cardiac allograft survival. The IL-4 and IL-10 combined gene therapy approach we developed recently is the only one successful gene therapy approach which could promote allograft tolerance without conventional immunosuppressive agents in large animals. We have developed a nonviral reporter-therapeutic linked ex vivo gene transfer technology for noninvasively and quantitatively monitoring cardiac transgene expressions in the whole heart using microPET. We used rabbit heart transplant/IL-10 gene therapy model as a tool validated the feasibility of this technology by systematically examining the reporter-therapeutic linked gene transfer efficiency and PET quantification accuracy in comparison to the findings of tissue analysis. Most importantly, using this model we validated and confirmed that PET reporter-therapeutic linked gene transfer dose not have local and systemic adverse effects, and reporter gene transfer dose not compromise the therapeutic efficacy of the co-transfected therapeutic gene. These questions are crucial in clinical application, but it was never been addressed in any of reporter gene studies previously. Thus, while we are further refining the quantification method, recently, we developed a coronary sinus retrograde reporter-therapeutic gene delivery technique for microPET imaging in vivo transfected gene expression in rabbits. Most recently, we have already shown the feasibility of monitoring the in vivo percutaneously coronary sinus retrograde delivered reporter-therapeutic linked transgene expression in canine heart using conventional PET scanner.

The fourth major component of the illustrated embodiment of the invention is a clinically applicable quantification method for monitoring the therapeutic transgene expression in targeted organs and tissues:

So far, the reporter gene expression was never been able to quantitatively analyzed and its correlation with the reporter probe accumulation in the heart was never examined. Although attempts have been made to inject an adenoviral vector with a reporter-VEGF linked gene into rat myocardium, the gene expression level was never been examined. The correlation between the reporter protein and VEGF protein expression assessed in cultured H9c2 cells using an in vivo quantification method has not been established. To date, in animal experiments, the quantification of signal for microPET imaging is still measured by the amount of tracer accumulated in a given tissue site normalized to the injected amount and to the mass of the tissue examined, % ID/g. Thus, animal has to be sacrificed. The heart tissue was collected and weighted. In vivo, the heart mass could only be assumed based on the total body weight.

In the illustrated embodiment of the invention we developed and validated the approach of consecutive PET imaging of [¹⁸F]-FHBG accumulation for quantifying reporter gene expression and [¹⁸F]-FDG accumulation for metabolic imaging of viable myocardium. We compared the correlation between the amount of trace accumulation assessed by % ID/g of [¹⁸F]-FHBG versus the ratio of % ID-[¹⁸F]-FHBG/% ID-[¹⁸F]-FDG to the transgene and protein expression levels assessed by ex vivo quantitative RT-PCR and Western blot analysis. The correlation of the HSV1-sr39tk gene and protein expression with the ratio of % ID-[¹⁸F]-FHBG/% ID-[¹⁸F]-FDG was significantly closer than that with % ID/g of [¹⁸F]-FHBG (r²=0.95 versus r²=0.92, P<0.05, and r²=0.94 versus r²=0.91, P<0.05, respectively). The correlation of the IL-10 gene and protein expression with the ratio of % ID-[¹⁸F]-FHBG/% ID-[¹⁸F]-FDG was also significantly stronger than that with % ID/g of [¹⁸F]-FHBG (r²=0.97 versus r²=0.88, P<0.05, and r²=0.95 versus r²=0.91, P<0.05, respectively).

Thus, it is to be understood that the illustrated embodiment of the invention is best practiced by long-term noninvasively, quantitatively and repeatedly monitoring the ex vivo targeted therapeutic transgene expression in various tissues and organs using reporter-therapeutic linked gene/probe with positron emission tomography, gamma camera or single-photon emission computed tomography.

The illustrated embodiment of the invention is also best practiced by long-term noninvasively, quantitatively and repeatedly monitoring the in vivo targeted therapeutic transgene expression in various tissues and organs using reporter-therapeutic linked gene/probe with positron emission tomography, gamma camera or single-photon emission computed tomography.

We have validated the efficiency, efficacy, and adverse effect of reporter therapeutic linked gene transfer in ex vivo gene therapy in hearts of rabbits and dogs. The toxicity has been examined in rabbits.

We also tested the efficiency, efficacy, and adverse effect of reporter therapeutic linked gene transfer in in vivo gene therapy in hearts of rabbits and dogs. Validation of this technique for gene therapy in other organs, such as joint, lung, liver, kidney, is contemplated.

In one embodiment the step of quantitatively imaging a reporter gene expression comprises the step of quantitatively imaging the expression of two or more reporter genes, which reporter genes are linked with two or more transfected gene-probes, to infer levels, location, or duration of the transfected gene expression in the targeted tissues, organs or cells.

In another embodiment the step of quantitatively imaging a reporter gene expression comprises the step of quantitatively imaging an expression of a reporter-therapeutic linked transgene vector induced by a balanced reporter/therapeutic or balanced reporter/diagnostic transgene expression with a bidirectional promoter located between a reporter gene and a therapeutic or diagnostic gene in a plasmid.

In yet another embodiment the step of quantitatively imaging a reporter gene expression comprises the step of quantitatively imaging an expression of a balanced reporter therapeutic transgene, or quantitatively imaging an expression of a proportional reporter and therapeutic or diagnostic transgene.

In one illustrated embodiment the step of quantitatively imaging a reporter gene expression comprises the step of quantitatively PET imaging transgene or ectopic transgene expression in targeted organs, tissues or cells.

In another one of the illustrated embodiments the step of quantitatively imaging a reporter gene expression comprises the step of quantitatively imaging a ratio of intensive transfection densities of an organ, tissue or cell by simultaneous measuring the expression of a metabolic probe and expression of a therapeutic or diagnostic transgene and ratioing the measurements.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1A is a diagram which shows the structure of the plasmid vector in which the promoters, reporter gene and therapeutic gene are linked.

FIG. 1B is a bar chart showing the efficiency of gene transfection.

FIG. 1C is a chart showing the results from an RT-PCR analysis of sr39tk and hIL-10 transgene expression in various organs and regions of the heart.

FIG. 1D is a bar chart showing the dose dependence of the transgene/GAPDH expression ratio for the reporter and therapeutic gene.

FIG. 1E is a graph showing the time dependence transgene/GAPDH expression ratio as a function of the number of postoperative days for linked genes vector and the “empty” liposome vector.

FIG. 2A is a graph showing the time dependence of the protein expression of the reporter and therapeutic genes as a function of the number of postoperative days.

FIG. 2B is a microphotograph showing the immunofluorescence staining which identifies the colocalization of the reporter and therapeutic genes.

FIG. 2C show the results of a Western blot analysis of the reporter and therapeutic genes for various locations in the heart shown above a bar chart of the protein expression for the reporter and therapeutic genes for the same locations in the heart.

FIG. 2D is a graph of the protein expression for the therapeutic as a function of the reporter gene showing the correlation between the two.

FIG. 3A is a series of microPET images of a rabbit heart taken at various numbers of postoperative days.

FIG. 3B is a graph of the time dependence of the myocardium % ID for 15 rabbits.

FIG. 3C is a series of microPET images from a rabbit's neck and chest of showing for the accumulation for a metabolic probe that in both donor heart in the neck and the rabbit's native heart in the chest as compared to a reporter-therapeutic linked transgene/[¹⁸F]FHBG probe that is only in the gene transfected transplanted donor heart in the neck, but not in the rabbit's native heart in the chest.

FIG. 4A is a series of tomographic PET images of a whole heart comparing [¹⁸F]FHBG images demonstrating the homogeneously distributed reporter-therapeutic gene expression and [¹⁸F]FDG images showing the viable myocardium.

FIG. 4B is a graph showing the correlation between [¹⁸F]FHBG accumulation (% ID/g) and ex vivo gamma counting of an explanted heart and Western blot quantification of TK protein expression level in the myocardium tissue.

FIG. 4C is a graph showing the correlation between IL-10 gene expression and [¹⁸F]FHBG/[¹⁸F]FDG ratio and [¹⁸F]FHBG accumulation (% ID/g) in the donor rabbit hearts.

FIG. 5A is a bar chart showing the mean survival of cardiac allografts as a function of days for various liposome complexed empty and reporter-therapeutic gene linked vector combinations.

FIG. 5B are histological microphotographs corresponding to the bar chart data points of FIG. 5A.

FIG. 5C is a graph of the rejection scores of the allografts of FIGS. 5A and 5B.

FIG. 5D is a bar chart showing the comparison in the CD3+ lymphocyte infiltration in the cardiac allografts reduced by liposome-pCMVhIL-10 gene therapy and by reporter-therapeutic linked gene therapy.

FIG. 5E is a bar chart showing the left ventricle systolic pressure for various cardiac allografts treated by different vector combinations compared with that in controls (allografts) and isografts.

FIG. 5F is a bar chart showing the number of incidents of arrhythmia in the allografts of FIG. 5E.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The illustrated embodiment is a clinically applicable approach for noninvasive monitoring of reporter and therapeutic linked gene expression in the whole heart of large animals using PET imaging. The efficacy and cardiac adverse effects of reporter and therapeutic linked gene transfer in a rabbit cervical heterotopic functional heart transplant model has been validated. Cationic liposome complexed with a vector containing a herpes simplex virus type 1 mutant thymidine kinase (HSV1-sr39tk) as the reporter gene and a recombinant human immunosuppressive cytokine, interleukin-10 (hIL-10), as the therapeutic gene was ex vivo intracoronarily delivered into cardiac allografts before implantation. Long-term HSV1-sr39tk and hIL-10 transgene and protein over expression associated with myocardial PET reporter probe 9-(4-[¹⁸F]fluoro-3-hydroxymethylbutyl)guanine ([¹⁸F]FHBG) accumulation was observed in the allografts. The expression of the HSV1-sr39tk gene was significantly correlated with the hIL-10 gene expression and the total myocardial [¹⁸F]FHBG accumulation quantified as a percentage of intravenously injected [¹⁸F]FHBG dose. A homogeneous distribution of [¹⁸F]FHBG accumulation was seen in the whole heart similar to the distribution of [¹⁸F]fluorodeoxyglucose, a PET glucose metabolism probe. The immunosuppressive therapeutic efficacy remained the same in allografts treated with reporter-therapeutic linked gene and therapeutic gene only. No cardiac adverse effect was found. Our results demonstrate for the first time that PET reporter-therapeutic linked gene imaging is applicable for noninvasively monitoring ex vivo intracoronarily delivered therapeutic transgene expression in the whole heart.

The illustrated embodiment is directed to a clinically applicable approach for ex vivo intracoronary delivery of a nonvirally mediated PET reporter-therapeutic linked transgene to the whole heart of a large animal. The accuracy of the reporter-therapeutic gene/probe PET imaging for noninvasively and quantitatively monitoring the distribution and kinetics of therapeutic transgene expression and examined the cardiac adverse effect and efficacy of reporter/immunosuppressive therapeutic gene therapy is validated using a rabbit heterotopic functional heart transplant model.

Before considering the results and validation of the methodology of the illustrated embodiment, consider the method which was performed as the illustrated embodiment. It must be expressly understood that many different modifications could be made in the illustrated methodology without departing from the scope of the invention.

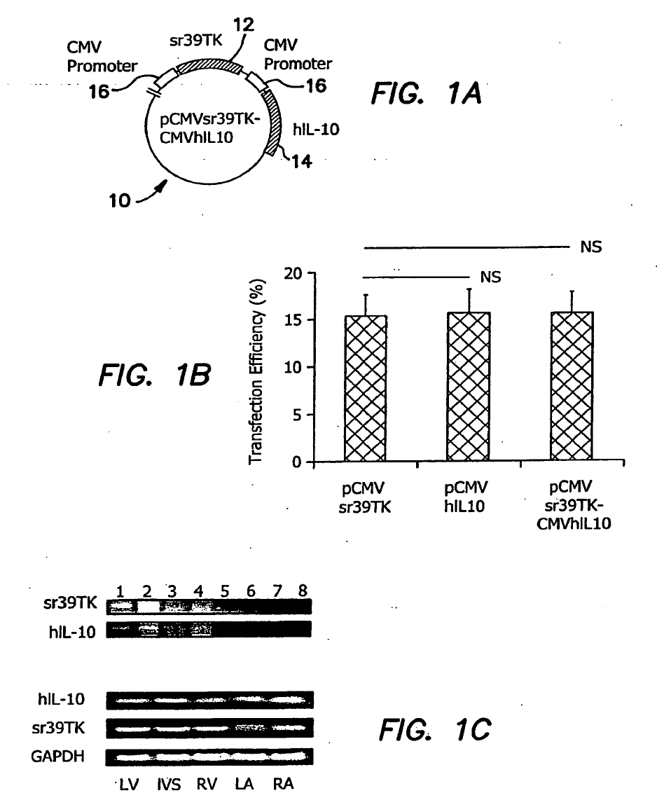

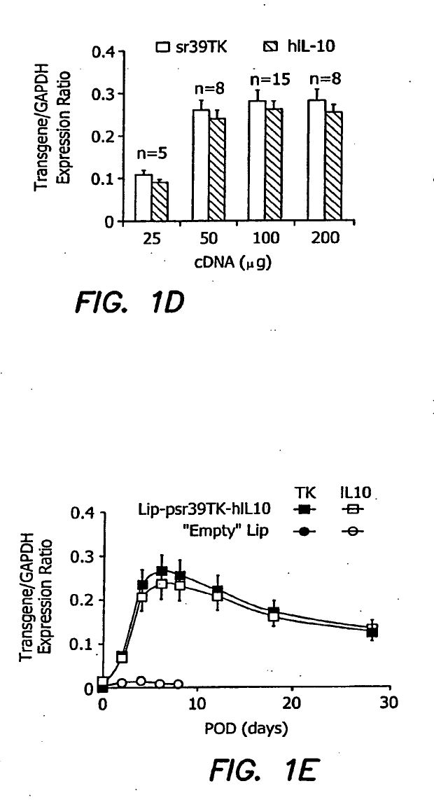

Turn first to the liposome-gene complex preparation. We constructed a plasmid vector 10 containing a mutant herpes simplex virus type 1 thymidine kinase gene (HSV1-sr39tk), as the PET reporter gene 12, and the human recombinant interleukin 10 gene, as therapeutic gene 14, which were driven by two identical CMV promoters 16 as illustrated diagrammatically in FIG. 1A. The cationic liposome GAP:DLRIE in the 2,3-dioxy-propaniminium class of cationic lipid basic skeleton, which also includes (+)-N-(2-hydroxyethyl)-N,N-dimethyl-2, 3-bis(tetradecyloxy)-1-propaniminium bromide (DLRIE), N-[1-(2,3-dioleyloxy) propyl]-N,N,N-trimethylammonium, 1,2-bis(oleoyloxy)-3-(trimethylammonio)propane, and 2,3-dioleyloxy-N-[2-(sperminecarboxamido)ethyl]-N,N-dimethyl-1-propanammonium, was provided by GIBCO BRL. The optimized concentration of pCMVHSV1-sr39tk, pCMV-hIL-10, or pCMV-HSV1-sr39tk-CMV-hIL-10 cDNA (50 μg) was complexed with liposome (50 μg) by 20 min gentle vortexing.

Heterotopic functional cervical heart transplantation model and ex vivo intracoronary gene delivery used was as follows. New Zealand White donor rabbits weighing 3.5 kg (Charles River Laboratories, St. Constant, QC, Canada) and recipient rabbits weighing 4 kg (Myrtle's Rabbitry, Thompson Station, Tenn., USA) were purchased from geographically unrelated vendors. The pathologic characteristics of this mismatch acute rejection model have been described previously. Briefly, under general anesthesia, donor rabbit hearts were arrested by infusion of University of Wisconsin solution (48 C, 20 ml/kg, 120 ml/h) through an aortic cannula. Liposome-gene complex in 10 ml normal saline was administrated by ex vivo intracoronary infusion in 20 min. Donor aorta and pulmonary artery were anastomosed to the recipient's proximal right carotid artery and common jugular vein, respectively, and the left and right atrium were anastomosed to the recipient's distal right carotid artery and common pulmonary artery, respectively.

Micro-PET scan was performed on postoperative days 2, 4, 6, 8, 12, 20, and 28 in the IL-10 gene therapy group and days 2, 4, 6, and 8 in the control group. Recipient rabbits were intravenously injected with 1 mCi of [¹⁸F]FHBG PET reporter probe. After a 1-h rest to allow for tracer uptake and clearance, the rabbits were imaged with a micro-PET-P4 (Primate P4; Concorde Microsystems, Inc., Knoxville, Tenn., USA) for 45 min over the neck and chest. Micro-PET image data were reconstructed by filtered back-projection and were reoriented into short, vertical, and horizontal long axis slices. From regions of interest on the anterolateral wall (short axis cut), derived counts/pixel/min were converted to counts/ml/min using a calibration constant obtained from scanning a cylindrical phantom. The regions-of-interest (ROI) counts/ml/min were converted to counts/g/min (assuming a tissue density of 1 g/ml) and divided by the injected dose to obtain an image ROI-derived [¹⁸F]FHBG percentage injected dose per gram of heart (% ID/g). The total myocardial accumulation of [¹⁸F]FHBG was corrected for background activity in each region of interest, summed for the 12 slices, and expressed as % ID=(total activity in 12 regions of interest (ROI), left ventricle (LV) (MBq)-total activity in 12 ROI BG (MBq))/injected dose (MBq)×100. The day before the [¹⁸F]FHBG scanning, 2-[¹⁸F]fluoro-2-deoxy-d-glucose (1 mCi) was injected into the ear vein of the rabbits. [¹⁸F]FDG scanning was performed for 1 h. Myocardium metabolic PET imaging was performed 60 min after [¹⁸F]FDG injection using the same procedure as that for [¹⁸F]FHBG.

Gamma counting of ¹⁸F radioactivity in the explanted heart was performed as follows. After micro-PET scans, explanted hearts were counted for ¹⁸F radioactivity in a gamma well counter (Cobra II Auto-Gamma, Packard). The total myocardial accumulation of [¹⁸F]FHBG was expressed as % ID.

In situ hybridization for evaluating the gene transfer efficiency was performed as follows. To determine the gene transfer efficiency, antisense and sense digoxigeninlabeled riboprobes (Boehringer Mannheim) of HSV1-sr39tk and hIL-10 mRNA were synthesized and used for in situ hybridization on paraffin sections as described previously. The gene transfer efficiency was determined as the percentage of blue-stained positive cells in total cardiac myocytes counted in 10 high-power microscopic fields (magnification×400) per section. Transgenes were expressed not only in the cardiac myocytes, but also in endothelial cells and vascular smooth muscle cells. Only those observation fields without vessel were used for analysis. The subsequent section stained with hematoxylin and eosin (H&E) was used to distinguish the cardiac myocytes from other cells.

Comparative reverse transcription-polymerase chain reaction (RT-PCR) analysis was performed as follows. Comparative reverse transcription-polymerase chain reaction was performed to detect the transgene expression of HSV1-sr39tk and hIL-10 in cardiac allografts using the primers and methods described previously. Three competitive templates (CT) were constructed, one each for HSV1-sr39tk, hIL-10, and the housekeeping gene GAPDH. The amplification product of each CT differs in size from the original cDNA product of 70-170 bp. To control for the efficiency of individual RT-PCR reactions from which sample cDNA templates were drawn, the same amplification technique was used first to measure the expression of the housekeeping gene GAPDH. Samples of HSV1-sr39tk and hIL-10 cDNA equivalent to 50 ng total RNA from each individual RT-PCR reaction product were diluted appropriately to contain equal concentrations of CT cDNA, normalized to the expression of GAPDH in the sample. For each particular gene, 5 μl of the normalized RT-PCR product was coamplified with a constant amount of the gene-specific CT DNA. The relative amounts of testing gene cDNA in the various samples were determined by comparing their respective sample ratios of testing gene cDNA/CT DNA multiplied by the constant amount of CT DNA used for the particular gene in the competitive template RT-PCR. In addition, all samples were normalized against the respective GAPDH cDNA/CT DNA ratio. This normalization controls for the quantity of cDNA loaded in all samples. PCR samples were run on 2% agarose gel. The intensity of ethidium bromide luminescence was measured using Eagle Sight 3.0 Software (Stratagene, La Jolla, Calif., USA) to obtain digital-image acquisition, processing, and analysis. This software provides an analysis of the relative densities of gel images, which represent two-dimensional arrays of pixels. Gel images were further analyzed and quantified using NIH Image 1.54.

Western blot analysis was performed as follows. Protein (100 A-μg) was subjected to SDS-polyacrylamide gel electrophoresis. The blot was incubated with a 1:1000 diluted mouse anti-human IL-10 monoclonal antibody (eBioscience, San Diego, Calif., USA) or 1:1000 diluted rabbit anti-thymidine kinase antibody (from M. E. Black, University of Washington, Seattle, Wash., USA) and then with goat anti-mouse or rat anti-rabbit IgG secondary antibody (Jackson Laboratories, West Grove, Pa., USA).

Double immunofluorescent staining for evaluating the distribution of the protein expression was performed as follows. Paraffin sections were blocked in 10% goat serum for 2 h. Slides were incubated with a 1:100 diluted mouse anti-human IL-10 monoclonal antibody (eBioscience) and 1:100 diluted rabbit antithymidine kinase antibody overnight. Slides were then incubated with goat anti-mouse IgG-FITC conjugated secondary antibody and rat antirabbit IgG-RPE conjugated secondary antibody (Southern Biotechnology, 1:100) for 90 min.

Histology analysis and rejection score of cardiac allografts was

Histology analysis and rejection score of cardiac allografts was performed as follows. Standard H&E staining was performed on the serial sections of LV tissue for histological evaluation. Rejection scores of cardiac allografts were determined based on the standardization of nomenclature in the diagnosis of heart rejection established by the International Society for Heart and Lung Transplantation.

Immunohistochemical staining for examination of infiltrating cells was performed as follows. The serial sections were incubated with biotinylated primary antibody against rabbit CD3 (Spring Valley Laboratories, Woodbine, Md., USA) for 1 h. Antibody-biotin conjugate was detected with an AutoProbe III kit (Biomed Corp., Foster City, Calif., USA) with H&E counterstaining.

Assessment of proarrhythmic effect of gene transfer and cardiac allograft function was performed as follows. To examine the proarrhythmic effect of gene transfer, ECG was continuously recorded for 1 h on the cardiac isograft and allograft at 24 h after cardiac allograft implantation. To assess cardiac function, the grafts from both control and treatment groups underwent transatrial catheterization immediately after ECG recording to quantify left and right ventricular pressures. The peak systolic pressure was determined using a BIOpac MP100 system (BIOpac, Inc., Santa Barbara, Calif., USA).

All data are expressed as means+a standard deviation. Paired or unpaired Student t test was performed to compare the difference between two groups. P<0.05 was regarded as significant.

The details of the methods used now having been explained turn and consider the results of the methods. First, consider the efficiency of ex vivo intracoronarily delivered and liposome-mediated reporter-therapeutic linked gene transfer. The efficiency of liposome-mediated ex vivo reporter-therapeutic linked gene transfer in cardiac allograft evaluated by in situ hybridization was moderate (15.5 and 15.4%, respectively). The gene transfer efficiency for the reporter gene was the same as for the therapeutic gene. It was also similar to that seen when either reporter or therapeutic gene was transferred alone (15.3 and 15.7%, respectively as shown in the bar graph of FIG. 1B. Efficiency of gene transfection is compared in FIG. 1B among the donor hearts transfected with reporter gene pCMVsr39tk alone (n=8), therapeutic gene pCMVhIL-10 alone (n=8), or reporter-therapeutic linked gene (pCMVsr39tk-CMVhIL-10, n=8) at postoperative day 8. Transgenes were expressed not only in the cardiac myocytes but also in endothelial cells and vascular smooth muscle cells with slightly higher gene transfer efficiency (18.8 and 17.1%, respectively).

Distribution and colocalization of reporter and therapeutic transgene expression analysis in the whole heart by RT-PCR and Northern blot revealed that both the PET reporter gene and the hIL-10 therapeutic gene were expressed only in the cardiac allografts, not in the recipients' heart, brain, lung, liver, kidney, or skeletal muscle as shown in the bar graph of FIG. 1C. Intracoronary gene delivery resulted in a colocalization and homogeneously distributed reporter and therapeutic gene over expression in whole heart FIG. 1C. The top portion of FIG. 1C show the representative data from quantitative RT-PCR analysis of sr39tk and hIL-10 transgene expression in LV of cardiac allografts (lanes 1 and 2) and recipient heart, lung, brain, liver, kidney, and skeletal muscle (lanes 3-8). The bottom portion of FIG. 1C shows the quantitative RT-PCR analysis of the over expressed reporter and therapeutic transgene homogeneously distributed in the left ventricle (LV), interventricular septum (IVS), right ventricle (RV), left atrium (LA), and right atrium (RA) of a cardiac allograft.

Consider the magnitude and time course of reporter and therapeutic transgene expression. The transgene expression in the targeted organ was dose dependent as shown in the bar graph of FIG. 1D. Dose dependence of sr39tk and hIL-10 transgene expression in cardiac allografts are summarized with a histogram in FIG. 1D. Cardiac tissue samples were collected on postoperative day (p.o.d.) 8. Quantitative sr39tk and hIL-10 transgene cDNA expression levels were plotted as a ratio to the expression of the housekeeping gene GAPDH (*P<0.05). We observed a parallel increase of the reporter and therapeutic transgene expression across the full dose range, except the highest dose. The significant increase in HSV1-sr39tk and hIL-10 transgene expression could be observed in the donor hearts as early as postoperative day 2, reached a peak at postoperative day 8, and then declined slowly as shown in the time graph of FIG. 1E. Time dependence of sr39tk and hIL-10 gene expression in allografts treated with reporter-therapeutic linked gene versus “empty” liposome were assessed by RT-PCR in FIG. 1E. To determine the time course of the transgene and protein expression, in the Lipsr39TKhIL-10-treated group animals were sacrificed and left ventricular tissue was collected for analysis at p.o.d. 0 (n=5), 2 (n=5), 4 (n=5), 6 (n=8), 8 (n=15), 12 (n=5), 18 (n=5), or 28 (n=5). In the control group treated with empty liposome, most allografts could survive for only 8 days; therefore animals were sacrificed and left ventricular tissue was collected for analysis at p.o.d. 0 (n=5), 2 (n=5), 4 (n=5), 6 (n=8), or 8 (n=15). The magnitude and time course of reporter gene expression induced by the reporter-therapeutic linked gene in the cardiac allografts were similar to those of the therapeutic transgene and were also the same as those of these two genes transferred separately.

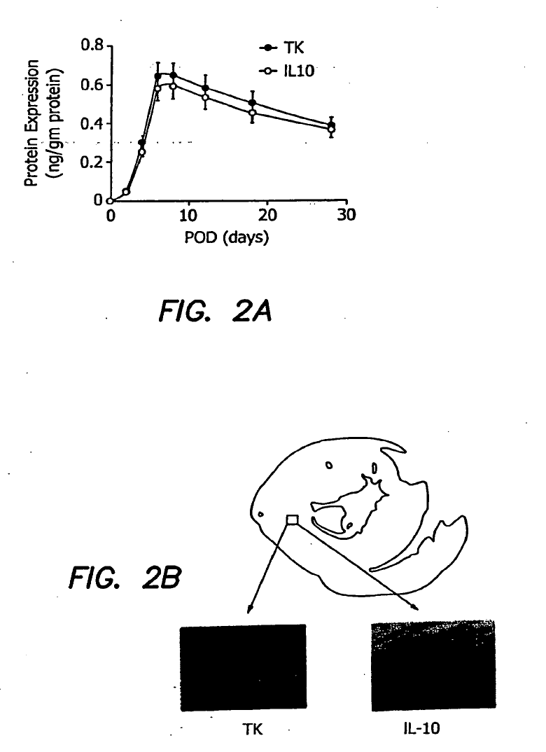

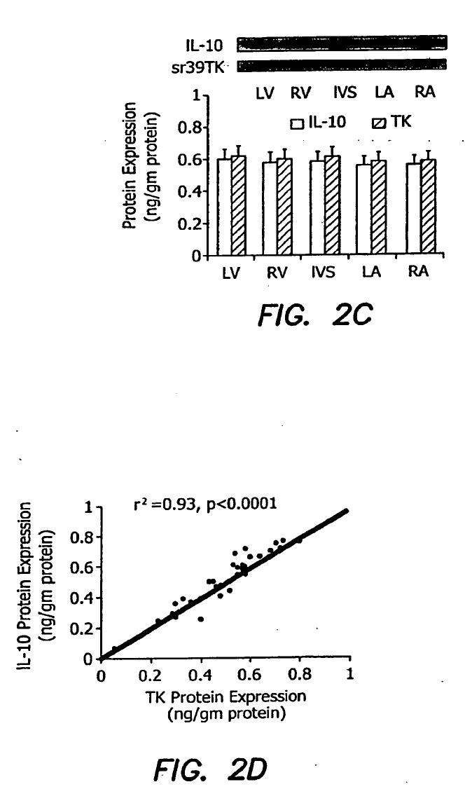

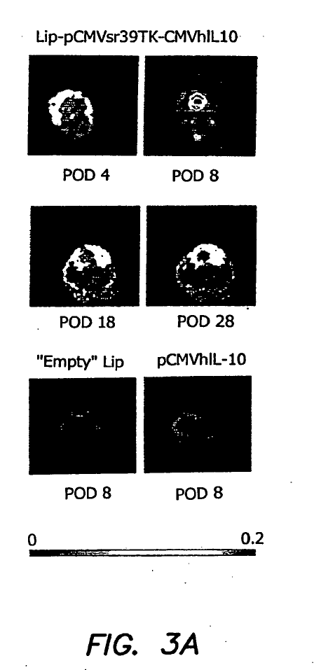

Consider the kinetics of balanced expression of the reporter and therapeutic gene products. Western blot analysis demonstrated that the time courses of the protein expression of both HSV1-sr39tk and hIL-10 were the same as those of gene expression as shown in FIG. 2A where the time dependence of TK and IL-10 protein over expression in cardiac allografts is depicted. Most importantly, the magnitude and time course of hIL-10 protein expression in the allograft were the same as for HSV1-sr39tk expression.

Consider the distribution and colocalization of the reporter and therapeutic gene products. Double immunofluorescence staining revealed the homogeneous distribution and colocalization of HSV1-sr39tk and IL-10 protein expression in the myocardium as shown in FIG. 2B which shows the results of immunofluorescence staining to identify the colocalization of TK and IL-10 protein expression in the cardiac allografts. Western blot analysis demonstrated that both HSV1-sr39tk and IL-10 protein levels are similar in the left atrium (LA), right atrium (RA), right ventricle (RV), interventricular septum (IVS), and left ventricle (LV) of donor heart as shown in FIG. 2C where the representative result of Western blot analysis shows the homogeneous distribution of over expressed TK and IL-10 protein in the LV, RV, IVS, LA, and RA. There was no significant change in HSV1-sr39tk and IL-10 concentration in the recipients' brain, lung, spleen, liver, kidney, or skeletal muscle in all time phases examined by ELISA compared with those recipient rabbits that had allografts treated with “empty” liposome as controls.

Correlation between reporter and therapeutic gene and protein expression is shown in FIG. 2D. The reporter gene expression in the cardiac allografts was significantly correlated with the therapeutic gene expression (r=0.94, P -b<0.001, data not shown). Additionally, HSV1-sr39tk protein expression was also very closely correlated with IL-10 protein expression in the targeted myocardium FIG. 2D where the correlation between TK and IL-10 protein levels in cardiac allografts transfected with reporter-therapeutic linked gene is unambiguously demonstrated.

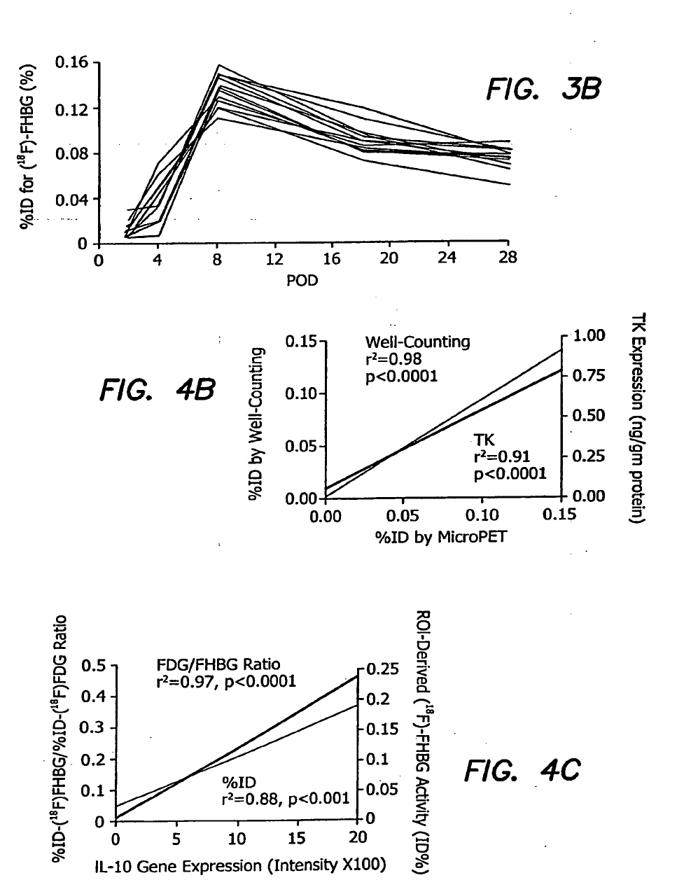

Micro-pet imaging of the reporter-therapeutic linked gene expression in the whole heart is graphically demonstrated. Here, for the first time, we have been able to show the homogeneous distribution of 9-(4-[¹⁸F]fluoro-3-hydroxymethylbutyl) guanine ([¹⁸F]FHBG) in the whole heart transfected with the reporter-therapeutic linked gene, pCMV-HSV1-sr39tk-CMVhIL-10 as depicted in images of FIG. 3A. Micro-PET imaging of [¹⁸F]FHBG accumulation in the myocardium of rabbit cardiac allografts is shown. Representative transaxial images of a donor rabbit cardiac allograft implanted in the neck of a recipient rabbit that was intracoronarily delivered ex vivo with liposome-pCMVsr39tk-CMVhIL-10. At p.o.d. 4, the distinct tracer accumulation was seen. At p.o.d. 8 a homogeneous distribution of [¹⁸F]FHBG accumulation in the myocardium of LV was observed. A decline of [¹⁸F]FHBG activity was seen at p.o.d. 18 and 28, while LV thickening occurred due to the acute allograft rejection. In contrast, [¹⁸F]FHBG accumulation was not observed in allografts treated with empty liposome or liposome-pCMVhIL-10 at p.o.d. 8. PET imaging of accumulated [¹⁸F]FHBG could be observed in pCMVHSV1-sr39tk-CMVhIL-10-treated cardiac allografts as early as p.o.d. 2, reached a peak at p.o.d. 8, maintained the high level at p.o.d. 12, and declined slowly as shown FIGS. 2A and 2B. The graph of FIG. 3B shows the time course of % ID for myocardial [¹⁸F]FHBG accumulation calculated from micro-PET images serially scanned in 15 rabbits. The clear signal of reporter probe activity was persistent out to p.o.d. 28. The kinetics of [¹⁸F]FHBG activity observed by long-term and repetitive noninvasive PET imaging was similar to that seen in transgene and protein measurements of tissue samples as seen in FIG. 1E. We did not see [¹⁸F]FHBG activity in empty liposome- or pCMVhIL-10-treated allografts. We observed [¹⁸F]FHBG activity only in targeted donor heart in the neck and not in the recipient's native heart nor other organs, such as lung, brain, skeletal muscle, or liver a shown in FIG. 3C which is the micro-PET imaging of localization of reporter-therapeutic linked transgene/[¹⁸F]FHBG probe accumulation in comparison with the metabolic probe, [¹⁸F]FDG, accumulation. In contrast, we observed significant 2-[¹⁸F]fluoro-2-deoxy-d-glucose ([¹⁸F]FDG) activity in the donor heart as well as in the recipient's heart and skeletal muscle of limb and neck in FIG. 3C.

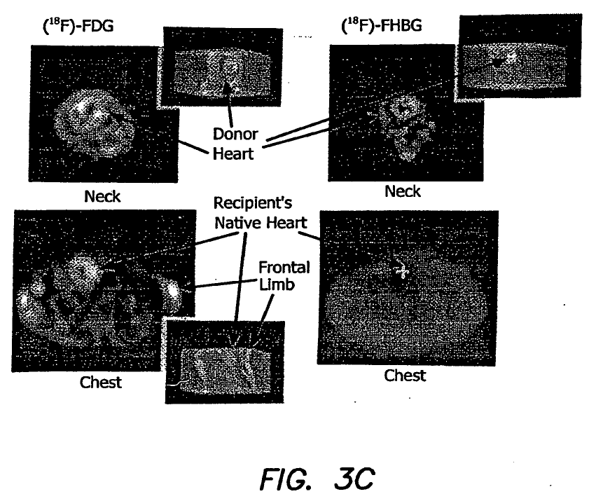

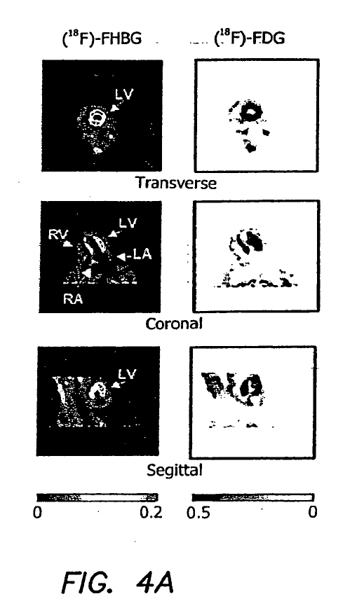

The homogeneous distribution of [¹⁸F]FHBG we saw in the donor heart was similar to the distribution shown by [¹⁸F]FDG shown in FIG. 4A, which is a tomographic view of whole heart micro-PET image. FIG. 4A are the [¹⁸F]FHBG images demonstrating the homogeneously distributed reporter-therapeutic gene expression in the whole heart and [¹⁸F]FDG images showing the viable myocardium. Color scale is expressed as % ID/g. Even though the gene transfer efficiency of liposome is five times lower than that of adenovirus, diffused distribution of [¹⁸F]FHBG activity was still clearly seen in RV and LV walls and IVS in the short, vertical, and horizontal axis images with this advanced high-resolution PET system.

Consider the quantification of therapeutic transgene expression. Traditional gamma counting of ¹⁸F radioactivity in LV from allografts treated with pCMV-HSV1-sr39tk-pCMVhIL-10 was 17 F 4-fold higher than that from allografts treated with pCMVhIL-10 or empty liposome. Ex vivo ¹⁸F gamma counting activity of explanted hearts was highly correlated with the ROI-derived micro-PET [¹⁸F]FHBG activity as shown in FIG. 4B, which shows the correlation between [¹⁸F]FHBG accumulation (% ID/g) and ex vivo gamma counting of explanted heart or Western blot quantification of TK protein expression level in the myocardium tissue. The total myocardial [¹⁸F]FHBG accumulation quantified as percentage of intravenously injected [¹⁸F]FHBG dose (% ID/g) was significantly correlated with HSV1-sr39tk mRNA level in myocardium (r²=0.83, P b 0.001, data not shown) and the HSV1-srt39TK protein level in FIG. 4B. The correlation between HSV1 sr39tk protein levels and FHBG % ID/g remained the same in allografts treated with pCMV-HSV1-sr39tk-pCMVhIL-10 or allografts treated with pCMVHSV1-sr39tk only. Most importantly, hIL-10 gene and protein expression levels were also highly correlated with [¹⁸F]FHBG accumulation (r²=0.88, P -b<0.001 and r²=0.91, P -b<0.001, respectively, FIG. 4C, which shows the correlation between IL-10 gene expression and [¹⁸F]FHBG/[¹⁸F]FDG ratio or [¹⁸F]FHBG accumulation (% intake dose (ID/g)) in the donor hearts. Quantitative imaging can be accomplished by measuring the radiographic density of the transfected reporter gene or molecule, like [¹⁸F]FDG, and measuring the radiographic density of a glucose marker molecule, like [¹⁸F]FHBG, in living tissue. The ratio of these two radiographic densities provides a quantitative measure of the amount of transfected material taken up per unit volume, per unit mass or per any other unitization measure of the organ, tissue or cell. In the in vivo instance there is no other practical way to obtain an accurate measure of volume or mass or an organ, tissue or cell since volume or mass sizes of organs, tissues and cells vary widely between different individuals or even between different locations within the body of a single individual. Marking the glucose uptake in living tissue and then ratioing that with the uptake of the transfected material, provides an accurate means of making a quantitative measurement or image of transfection densities.

To date, the quantification of signal for micro-PET imaging is still measured by the amount of tracer accumulated in a given tissue site normalized to the injected amount and to the mass of the tissue examined, % ID/g. In vivo, the heart mass could only be assumed based on the total body weight. Here we validated the approach of consecutive PET imaging of [¹⁸F]FHBG accumulation for quantifying reporter gene expression and [¹⁸F]FDG accumulation for metabolic imaging of viable myocardium. We compared the correlation between the amount of trace accumulation assessed by % ID/g of [¹⁸F]FHBG versus the ratio of % ID [¹⁸F]FHBG/% ID [¹⁸F]FDG to the transgene and protein expression levels assessed by ex vivo quantitative RT-PCR and Western blot analysis. The correlation of the HSV1-sr39tk gene and protein expression with the ratio of % ID [¹⁸F]FHBG/% ID [¹⁸F]FDG was significantly closer than that with % ID/g of [¹⁸F]FHBG (r²=0.95 versus r²=0.92, P -b<0.05, and r²=0.94 versus r²=0.91, P -b<0.05, respectively). The correlation of the IL-10 gene and protein expression with the ratio of % ID [¹⁸F]FHBG/% ID [¹⁸F]FDG was also significantly stronger than that with % ID/g of [¹⁸F]FHBG (r²=0.97 versus r²=0.88, P -b<0.05, and r²=0.95 versus r²=0.91, P -b<0.05, respectively, in FIG. 4C).

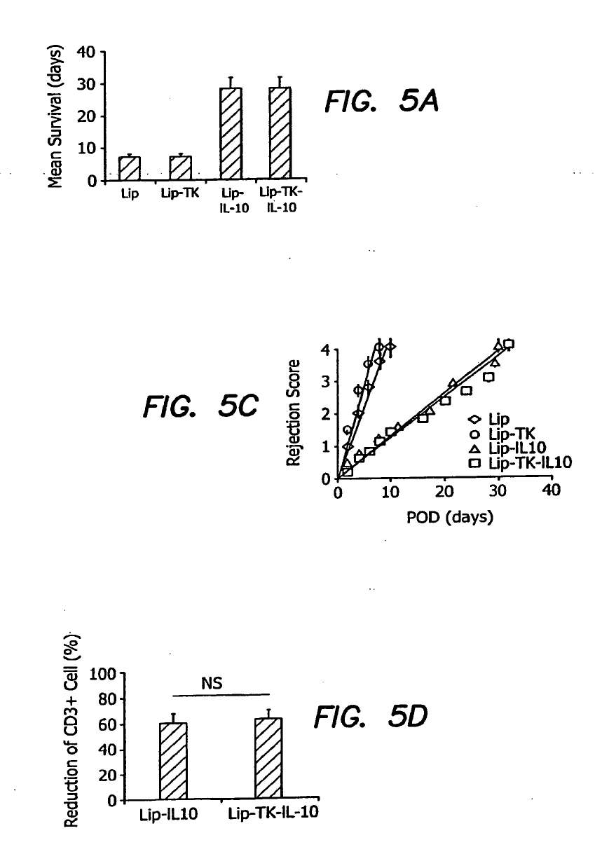

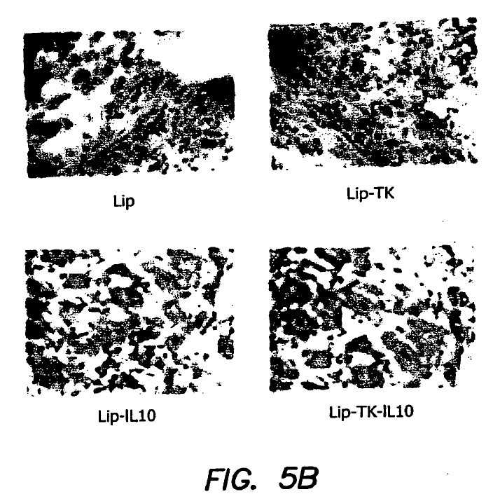

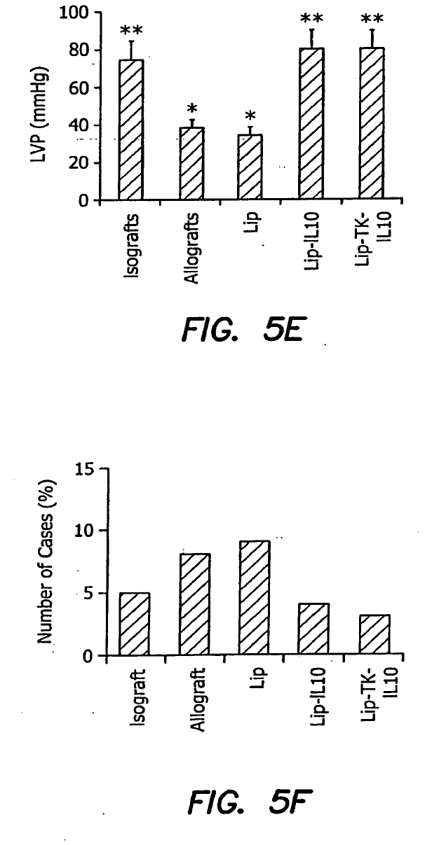

Consider the therapeutic efficacy and adverse effect of reporter-therapeutic linked gene transfer. Reporter-therapeutic linked gene transfer did not affect RV and LV systolic pressure or heart rate in isografts (heart transplant was performed on third generation of copulating sibling New Zealand rabbits) during 2 h of monitoring at p.o.d. 2, 4, 6, 8, 12, 20, and 28. No significant proarrhythmic effect was found. We further examined whether transfection of a reporter-therapeutic linked transgene could compromise the efficacy of immunosuppressive gene therapy. The survival of cardiac allografts was increased from 7±1 days in allografts treated with empty liposome or pCMV-HSV1-sr39tk to 28±7 days in allografts treated with pCMV-HSV1 sr39tkpCMVhIL-10 or pCMVhIL-10 in the bar graph of FIG. 5A. FIGS. 5A-5F show the effects of reporter-therapeutic linked gene transfection in cardiac allografts on cardiac function and the efficacy of gene therapy. FIG. 5A is a bar chart of the comparison of mean survival in cardiac allografts treated with empty liposome (Lip; n=15), liposome-pCMVsr39tk (Lip-TK; n=15), liposome-pCMVhIL-10 (Lip-IL-10; n=15), or liposome-pCMVsr39tk-CMVhIL-10 (Lip-TK-IL-10; n=15). The rejection score was also improved to the same extent in the allografts treated with therapeutic gene linked or not to the reporter gene in the microphotographs of FIG. 5B, which are representative histological findings (H&E staining) in left ventricular tissue of cardiac allografts treated with empty liposome (Lip), liposome-pCMVsr39tk (Lip-TK), liposome-pCMVhIL-10 (Lip-IL10), or liposome-pCMVsr39tk-CMVh IL-10 (Lip-TK-IL10) at p.o.d. 8. We found the same degree of lymphocyte infiltration reduction in allografts treated with reporter-therapeutic linked gene versus therapeutic gene alone the graph of FIG. 5C, which is a bar graph of the comparison of the rejection score in the cardiac allografts affected by the empty liposome (Lip; n=15), liposome-pCMVsr39tk (Lip-TK; n=15), liposome-pCMVhIL-10 (Lip-IL10, n=15), or liposome-pCMVsr39tk-CMVhIL-10 (Lip-TK-IL10; n=15). We found improvement of LV systolic pressure to the same extent in both therapeutic gene- and reporter-therapeutic linked gene treated allografts in the bar graph of FIG. 5D, which is a bar chart of the comparison of the CD3+ lymphocyte infiltration in the cardiac allografts reduced by liposome-pCMVhIL-10 gene therapy and reporter-therapeutic linked gene therapy. We observed no proarrhythmic effect in the reporter-therapeutic gene transferred group in the bar graph of FIG. 5E, which is a bar chart of the comparison of LV systolic pressure in cardiac allografts treated with empty liposome (Lip; n=15), liposome-therapeutic gene only (Lip-IL-10; n=15), or reporter-therapeutic linked gene therapy (Lip-TK-IL-10; n=15) to that in cardiac isografts (n=15, *P -b<0.05) and allografts without any treatment (n=15, **P -b<0.05). Systolic pressure was recorded at p.o.d. 4. FIG. 5F is a bar chart of the incidence of arrhythmia that includes supraventricular tachycardia, atrial flutter and fibrillation, and ventricular tachycardia and fibrillation in the cardiac allografts treated with empty liposome (Lip; n=15), liposome-therapeutic gene only (Lip-IL10; n=15), reporter-therapeutic linked gene therapy (Lip-TK-IL10; n=15), cardiac isografts (n=15), and allografts without any treatment (n=15) at 24 h after commencement of reperfusion. ECG was recorded continuously for 1 h. Values are expressed as a percentage of total cases.

It can now be appreciated in view of the foregoing results that our results demonstrate for the first time that PET reporter gene imaging is applicable to noninvasive monitoring and quantifying of intracoronarily delivered therapeutic transgene expression in whole heart. Constructing a vector that is able to carry out a balanced and closely correlated dual gene expression is essential for this approach. Although previously the bicistronic approach using an internal ribosomal entry site (IRES) showed a good correlation of two PET reporter genes carried by adenovirus, the transgene expression downstream of the IRES was often attenuated. The bidirectional transcriptional approach utilized a vector in which the therapeutic and reporter genes were each driven by the cytomegalovirus (CMV) promoter containing a tetracycline-responsive element; however, a fusion protein was needed for coexpression. In the present study the vector containing a reporter and a therapeutic gene driven by two identical promoters was able to induce a balanced and colocalized reporter and therapeutic transgene expression in the targeted myocardium. A thorough evaluation confirmed that the linkage of reporter and therapeutic genes in one plasmid did not alter the transfer efficiency of either gene and is feasible for the indirect imaging of therapeutic gene expression. In contrast, when we used two different promoters, the expression level of reporter gene driven by the CMV promoter was higher than the therapeutic gene driven by the SV40 promoter (unpublished observation).

The close correlation between the reporter and the therapeutic gene and protein expression, in addition to the strong correlation between the reporter gene expression and the reporter probe activity, lays the foundation for the indirect monitoring of therapeutic gene expression by imaging the linked reporter gene with a PET reporter probe. In a previous study, viral titer correlated poorly with the accumulation of two cotransfected reporter probes, thought to be due to the variable adenoviral trafficking. Liposome-mediated gene transfer induces a stable and homogeneous transgene expression in the targeted organ that may also play a role in the superior correlation between the transgene/protein expression and the reporter probe accumulation.

With the whole-heart PET reporter probe accumulation image, PET imaging of the cardiac perfusion is no longer required for locating the position of the heart. However, [¹⁸F]FDG accumulation represents the viable myocytes that have better transcriptional and translational function. The ratio of [¹⁸F]FHBG/[¹⁸F]FDG represents the proportion of the transfected cells in the total viable myocytes and can be used for the quantification of true gene transfer efficiency. The superior correlation of the [¹⁸F]FHBG/[¹⁸F]FDG ratio with reporter and therapeutic gene and protein expression in the myocardium suggests advantages over the standard uptake value that is normalized by body weight (% ID/g).

The low signal of reporter transgene image due to the five times lower gene transfer efficiency of liposome compared with adenovirus was the major burden for PET imaging. The Micro-PET-P4 system used in this study, designed for rabbit and primate PET scanning, not only has approximately four times the axial field of view compared to the previous micro-PET system, but also has four times higher sensitivity, which remedies the low efficiency of liposome. On the other hand, in the present study intracoronary gene delivery induced a diffused distribution of reporter and therapeutic gene. This is another reason for the generally low signal compared to intramyocardially injected reporter gene carried by adenovirus, which was concentrated around the needle side and generates high signal in a very small region in all of the previous studies. Nevertheless, homogeneously distributed transgene/protein expression and [¹⁸F]FHBG accumulation in whole heart greatly accelerate the clinical application of noninvasive and quantitative PET imaging of therapeutic gene expression.

Liposome-mediated reporter-therapeutic linked gene transfer eliminates the virally induced autoimmune response and allows us to validate the possibility of long-term PET reporter gene/probe imaging. The immunogenicity of reporter gene products has always been a concern, but it was never confirmed because of the use of viral vector or the plasmid—DNA itself. We show that the liposome-mediated reporter-therapeutic linked gene transfection has the same magnitude and kinetics as therapeutic gene alone. The CD3+ infiltration remained the same in these two groups. All these findings suggest that the adenovirus, not the reporter gene products, is the cause of the autoimmune response and the transient reporter gene expression. Our results also indicated that the reporter gene and its product do not have significant adverse effect in cardiac isografts and allografts. Transferring the coupled reporter and therapeutic gene did not compromise the therapeutic efficacy of IL-10-induced immunosuppression in the rabbit heterotopic functional cardiac allograft transplant model.

As a major immunosuppressive cytokine and anti-inflammatory agent, IL-10 holds potential for the treatment of allograft rejection. However, systemic administration of IL-10 after transplantation did not show any benefits, mainly due to the significant pleiotropic effects, especially its immunostimulatory effect on B cells and activated CD8+ T cells. Previous studies in rodents and rabbits have shown that localized expression of recombinant IL-10 gene in the transplanted heart may contribute to the prevention and treatment of major problems in transplantation, such as acute rejection and accelerated allograft coronary atherosclerosis. Procured organs lend themselves readily to genetic engineering due to the technical requirement of temporary ex vivo preservation. The period of time between harvest and implantation of cardiac transplants provides a unique opportunity for ex vivo intracoronary delivery of the therapeutic gene(s) to modify the graft biologically and paves the way for local or organ-specific immunosuppression, specifically while avoiding systemic side effects and the need for conventional systemic immunosuppression. Previous studies have shown that the gene transfer efficiency in ex vivo intracoronary gene delivery was three to five times higher than in vivo intracoronary gene delivery. A great systemic leakage in in vivo gene delivery is responsible for the low local therapeutic gene expression, high ectopic gene transfection, and lack of success in clinical trials. Although the efficiency of liposome-mediated ex vivo intracoronary gene delivery is still relatively low, soluble IL-10 is homogeneously distributed in whole heart. The efficacy of over expressed IL-10 in allografts was higher than was seen in adenovirus-mediated ex vivo gene transfer and the action lasted much longer. Especially, the approach of 20 min ex vivo intracoronary gene delivery during organ procurement is clinically applicable. The noninvasive reporter-therapeutic linked transgene PET imaging system reported here could play a pivotal role in further validating the pharmacokinetics and pharmacodynamics of immunosuppressive cytokine or other therapeutic gene therapy in heart transplantation in large animals.

In conclusion, noninvasively quantifying reporter-therapeutic gene expression and viable myocardium completed a truly noninvasive transgene quantification system for potentially characterizing the pharmacokinetics of human gene therapy. Most importantly, the safety profile of this ex vivo intracoronary nonviral vehicle-mediated localized PET reporter-therapeutic gene targeting approach offers a significant advantage for clinical application.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An improvement in a method for noninvasively monitoring the expression of transgene ex vivo and/or in vivo delivery comprising quantitatively imaging a reporter gene expression, which reporter gene is linked with a transfected gene, to infer levels, location, or duration of the transfected gene expression in the targeted tissues, organs or cells.

2. The improvement of claim 1 where quantitatively imaging the reporter gene expression, which reporter gene is linked with the transfected gene, comprises linking the reporter gene to the transfected gene on a plasmid vector, in a cell or on DNA.

3. The improvement of claim 1 where quantitatively imaging the reporter gene expression, which reporter gene is linked with the transfected gene, comprises quantitatively imaging the reporter gene expression which is linked to a therapeutic gene.

4. The improvement of claim 1 where quantitatively imaging the reporter gene expression, which reporter gene is linked with the transfected gene, comprises quantitatively imaging the reporter gene expression which is linked to a diagnostic gene.

5. The improvement of claim 1 where quantitatively imaging the reporter gene expression, which reporter gene is linked with the transfected gene, comprises quantitatively imaging the reporter gene expression which is linked to an identical gene used as the therapeutic gene.

6. The improvement of claim 1 where quantitatively imaging the reporter gene expression, which reporter gene is linked with the transfected gene, comprises quantitatively imaging the reporter gene expression which is linked to a nonidentical gene used as the therapeutic gene.

7. The improvement of claim 1 where quantitatively imaging the reporter gene expression, which reporter gene is linked with the transfected gene, comprises quantitatively imaging reporter gene expressions of more than one reporter gene.

8. The improvement of claim 1 where quantitatively imaging the reporter gene expression, which reporter gene is linked with the transfected gene, comprises using a radiopharmaceutical for scintigraphic imaging of gene expression interactions with the reporter gene.

9. The improvement of claim 1 where quantitatively imaging the reporter gene expression, which reporter gene is linked with the transfected gene, comprises quantitatively imaging by positron emission tomography, gamma camera or single-photon emission computed tomography.

10. The improvement of claim 1 further comprising providing a reporter-therapeutic linked transgene vector with balanced reporter/therapeutic transgene expression.

11. The improvement of claim 10 where providing a reporter-therapeutic linked transgene vector with balanced reporter/therapeutic transgene expression comprises providing herpesviral thymidine kinase (HSV1-tk) with two identical cytomegalovirus (CMV) promoters with one reporter gene and one therapeutic gene in a single plasmid.

12. The improvement of claim 10 where providing a reporter-therapeutic linked transgene vector with balanced reporter/therapeutic transgene expression comprises providing two or more identical or nonidentical promoters with one reporter gene and two or more therapeutic genes in a single plasmid.

13. The improvement of claim 1 further comprising using liposome encapsulation to reduce the immune response, and to increase the efficiency of reporter and therapeutic linked gene transfer.

14. The improvement of claim 10 where providing a reporter-therapeutic linked transgene vector with balanced reporter/therapeutic transgene expression comprises providing two identical EF-1α promoters with one reporter gene and two or more therapeutic genes.

15. The improvement of claim 1 where quantitatively imaging a reporter gene expression comprises utilizing FHBG for in vivo imaging of the wild-type HSV1-tk and HSV1-sr39tk PET reporter genes.

16. The improvement of claim 1 further comprising providing a reporter-therapeutic linked transgene vector with balanced reporter/therapeutic transgene expression and where providing a reporter-therapeutic linked transgene vector with balanced reporter/therapeutic transgene expression comprises providing a cationic liposome complexed with a vector containing a herpes simplex virus type 1 mutant thymidine kinase (HSV1-sr39tk) as the reporter gene and a recombinant human immunosuppressive cytokine, interleukin-10 (hIL-10) as the therapeutic gene.

17. The improvement of claim 16 further comprises including a PET reporter probe 9-(4-[18F]fluoro-3-hydroxymethylbutyl)guanine ([18F]FHBG) with the reporter gene.

18. A transgenic composition for noninvasively monitoring the expression of transgene ex vivo and/or in vivo delivery comprising:
a reporter gene;
a gene-probe included in the reporter gene capable of imaging with positron emission tomography, a gamma camera or single-photon emission computed tomography; and
at least one transfected gene linked to the reporter gene.

19. The composition of claim 18 where the reporter gene linked to the transfected gene is on a plasmid vector, in a cell or on DNA.

20. The composition of claim 18 where quantitatively imaging the reporter gene expression, which reporter gene is linked with the transfected gene, comprises quantitatively imaging the reporter gene expression which is linked to a therapeutic gene.

21. The composition of claim 18 where quantitatively imaging the reporter gene expression, which reporter gene is linked with the transfected gene, comprises quantitatively imaging the reporter gene expression which is linked to a diagnostic gene.

22. The composition of claim 18 where quantitatively imaging the reporter gene expression, which reporter gene is linked with the transfected gene, comprises quantitatively imaging the reporter gene expression which is linked to an identical gene used as the therapeutic gene.

23. The composition of claim 1 where quantitatively imaging the reporter gene expression, which reporter gene is linked with the transfected gene, comprises quantitatively imaging the reporter gene expression which is linked to a nonidentical gene used as the therapeutic gene.

24. The composition of claim 18 where quantitatively imaging the reporter gene expression, which reporter gene is linked with the transfected gene, comprises quantitatively imaging reporter gene expressions of more than one reporter gene.

25. The composition of claim 18 where the gene probe comprises a radiopharmaceutical for scintigraphic imaging of gene expression interactions with the reporter gene.

26. The composition of claim 18 further comprising a reporter-therapeutic linked transgene vector with balanced reporter/therapeutic transgene expression.

27. The composition of claim 19 where the reporter-therapeutic linked transgene vector comprises herpesviral thymidine kinase (HSV1-tk) with two identical cytomegalovirus (CMV) promoters in a single plasmid.

28. The composition of claim 19 where the reporter-therapeutic linked transgene vector comprises two identical or nonidentical promoters and where the composition further comprises two or more therapeutic genes in a single plasmid.

29. The composition of claim 18 further comprising a liposome encapsulation to reduce the immune response, and to increase the efficiency of reporter and therapeutic linked gene transfer.

30. The composition of claim 19 where the reporter-therapeutic linked transgene vector further comprises providing two identical EF-1α promoters with one reporter gene and two or more therapeutic genes.

31. The composition of claim 18 further comprising a cationic liposome complexed reporter-therapeutic linked transgene vector with balanced reporter/therapeutic transgene expression including a herpes simplex virus type 1 mutant thymidine kinase (HSV1-sr39tk) as the reporter gene and a recombinant human immunosuppressive cytokine, interleukin-10 (hIL-10) as the therapeutic gene.

32. The composition of claim 18 further comprising a cationic liposome complexed with a vector and including a PET reporter probe 9-(4-[18F]fluoro-3-hydroxymethylbutyl)guanine ([18F]FHBG) with the reporter gene.

33. The improvement of claim 1 where quantitatively imaging a reporter gene expression comprises quantitatively imaging the expression of two or more reporter genes, which reporter genes are linked with two or more transfected gene-probes, to infer levels, location, or duration of the transfected gene expression in the targeted tissues, organs or cells.

34. The improvement of claim 1 where quantitatively imaging a reporter gene expression comprises quantitatively imaging an expression of a reporter-therapeutic linked transgene vector induced by a balanced reporter/therapeutic or balanced reporter/diagnostic transgene expression with a bidirectional promoter located between a reporter gene and a therapeutic or diagnostic gene in a plasmid.

35. The improvement of claim 1 where quantitatively imaging a reporter gene expression comprises quantitatively imaging an expression of a balanced reporter therapeutic transgene, or quantitatively imaging an expression of a proportional reporter and therapeutic or diagnostic transgene.

36. The improvement of claim 1 where quantitatively imaging a reporter gene expression comprises quantitatively PET imaging transgene or ectopic transgene expression in targeted organs, tissues or cells.

37. The improvement of claim 1 where quantitatively imaging a reporter gene expression comprises quantitatively imaging a ratio of intensive transfection densities of an organ, tissue or cell by simultaneous measuring the expression of a metabolic probe and expression of a therapeutic or diagnostic transgene and ratioing the measurements.
